(19) United States
(12) Patent Application Publication
(10) Pub. No.: US 2010/0055042 A1
(43) Pub. Date: Mar. 4, 2010

(54) Enhancing Clot Busting Medication in Stroke with Directed Drug Convection using Magnetic Nano-Particles

(76) Inventors: Vinith Yathindranath, Winnipeg (CA); Torsten Hegmann, Winnipeg (CA); Johan van Leirop, Winnipeg (CA); David F. Moore, Winnipeg (CA)

Correspondence Address: ADE & COMPANY INC., 2157 Henderson Highway, WINNIPEG, MB, R2G1P9, CA

(21) Appl. No.: 12/471,424

(22) Filed: May 25, 2009

Related U.S. Application Data
(60) Provisional application No. 61/055,683, filed on May 23, 2008.

(51) Int. Cl. A61K 47/02; B05D 7/00; A61K 49/08; A61K 9/14

(52) U.S. Cl. 424/9.36; 427/215; 427/221; 514/769; 424/490

(57) ABSTRACT

An important step towards successful drug targeting with nanoparticles is developing a method to coat the nanoparticles with a useful drug. BSA was used by us to mimic the actual drug for its cost effectiveness during initial trials. We have successfully immobilized BSA onto three (PEG, PEMA and glutamic acid) out of four different surfactant capped nanocomposites. We found that the BSA immobilized particles showed excellent colloidal stability in water and stayed well suspended without any sign of agglomeration or settling. The suspended particles were easily accumulated using a magnet and could be re-dispersed readily. These properties indicate that the BSA immobilized iron oxide nanocomposites are excellent candidates for directed drug convection (DDC).

### PRIOR APPLICATION INFORMATION

The instant application claims the benefit of U.S. Provisional Patent Application 61/055,683, filed May 23, 2008.

### BACKGROUND OF THE INVENTION

Magnetic nanoparticles can be influenced by an applied magnetic field. Attaching surfactant molecules to the surface of these nanoparticles produces nanocomposites that are magnetic. At the nanoscale, the relative surface area of the particles is high as compared to the bulk materials, and hence the loading capacity with respect to surfactant molecules and drug on the surface is higher. Iron oxide nanoparticles and their nanocomposites are of interest as they are highly magnetic and can be surface modified without much difficulty. Many studies have been reported on such iron oxide nanocomposites due to their importance in biomedical applications such as a MRI contrast agent¹, heating mediators for cancer thermotherapy², and as drug carrier systems³.

Developing an iron oxide nanocomposite for magnetically guided drug delivery is of interest as it enables target specificity and reduced drug dosage. To serve as the most useful drug carrier system, the particles should overcome crucial physiological barriers in vivo. To increase the effective drug carrying life of the nanocomposites in the blood stream and to escape phagocytosis by macrophages⁴, the particles should have prolonged stability in aqueous solutions (i.e., they need to be hydrophilic). Agglomeration of these particles should also be prevented inside the blood stream. Suitable surfactant molecules capable of tuning the surface properties can be used to achieve these requirements. As will be appreciated by one of skill in the art, tuning of the surface properties here means using suitable surfactant molecules to improve the stability and diffusion of the particles in aqueous medium (as required for drug carrier). Other long chain aliphatic surfactant molecules (for example, oleic acid or oleylamine) can be used to make the particles stable in organic medium (organic solvent) as required for other applications such as rotary shaft sealing, oscillation damping and position sensing Many studies have been reported on methods of coating iron oxide nanoparticles with biocompatible polymers⁵, proteins⁶ and other organic molecules⁷. These works have shown that a thorough understanding of the surface properties, the colloidal stability in aqueous medium, and the magnetic properties of the surface modified nanocomposites are crucial for successful magnetically guided drug delivery in biological systems.

Some of the polymers like the polyacrylates can immobilize proteins. M. Okubo et al.^{6a} reported a detailed study on the absorption of bovine serum albumin (BSA) on PHEMA/PS (polystyrene) composites. Pan et al. have immobilized BSA on dendrimer coated magnetite nanoparticles.^{6a} In this work, we have used the biocompatible surfactants polyethylene glycol (PEG), glutamic acid, poly(ethyl methacrylate) [PEMA] and poly(2-hydroxyethyl methacrylate) [PHEMA] to coat the iron oxide nanoparticles. The coated nanocomposites were characterized using powder x-ray diffraction (XRD), high-resolution transmission electron microscopy (HR-TEM), scanning electron microscopy (SEM), Fourier transform infrared spectroscopy (FT-IR), magnetometry, and Mössbauer spectroscopy.

### SUMMARY OF THE INVENTION

According to a first aspect of the invention, there is provided a method of preparing surface modified ion oxide nanocomposites comprising:

dissolving FeCl₃.6H₂O and FeCl₂.4H₂O in heated water comprising a suitable biocompatible polymer;

adding ammonium hydroxide and mixing, thereby producing a mixture;

cooling the mixture to about room temperature; and

recovering surface modified iron oxide nanocomposites from the mixture.

The suitable biocompatible polymer may be selected from the group consisting of polyethylene glycol (PEG), poly(ethyl methacrylate) (PEMA) and glutamic acid.

The surface modified iron oxide nanocomposites may have a diameter of approximately 7-20 nanometers.

The FeCl₃.6H₂O and the FeCl₂.4H₂O may be mixed at approximately a 2:1 ratio.

The water may be heated to about 80° C. in a further aspect of the invention, there are provided the additional steps of adding a drug to the recovered surface modified iron oxide nanocomposites, stirring and recovering drug coated surface modified iron oxide nanocomposites.

According to a further aspect of the invention, there is provided a surface modified iron oxide nanocomposited prepared according to the method described above.

### BRIEF DESCRIPTION OF THE DRAWINGS

FIG. 1: Sample vials from left to right: Iron oxide nanoparticle, PEMA capped iron oxide, PHEMA capped iron oxide, glutamic acid capped iron oxide, PEG capped iron oxide, PEG capped iron oxide with immobilized BSA, and BrMPA capped iron oxide with immobilized BSA. (a) Samples photographed directly after dispersion by sonication, and (b) after one week.

FIG. 2: PEMA capped iron oxide nanocomposite attracted to the wall of sample vial within a few seconds in the presence of a magnet.

FIG. 3: XRD pattern of bare iron oxide (Fe₃O₄/γ-Fe₂O₃) nanoparticles.

FIG. 4: HR-TEM images PEG capped (a, b), BrMPA capped (c), and PEMA capped iron oxide nanocomposites (d).

FIG. 5: SEM images of iron oxide nanocomposites surface modified with (a) glutamic acid, (b) PEG, (c) PEMA, and (d) PHEMA.

FIG. 6: FT-IR spectra of: (a) Bare iron oxide nanoparticles, (b) PHEMA capped iron oxide, (c) PEMA capped iron oxide, (d) PEG capped iron oxide, (e) glutamic acid capped iron oxide and (f) PEG capped iron oxide nanocomposites with immobilized BSA.

FIG. 7: Room temperature magnetization curve of surface modified iron oxide nanocomposites: a) weight normalized with respect to γ-Fe₂O₃ and b) weight normalized with respect to Fe₃O₄.

FIG. 8: Mössbauer spectra of a) bare iron oxide nanoparticles and b) PEMA grafted nanocomposite.

FIG. 9: Synthesis of PEMA and PHEMA capped iron oxide nanocomposite.

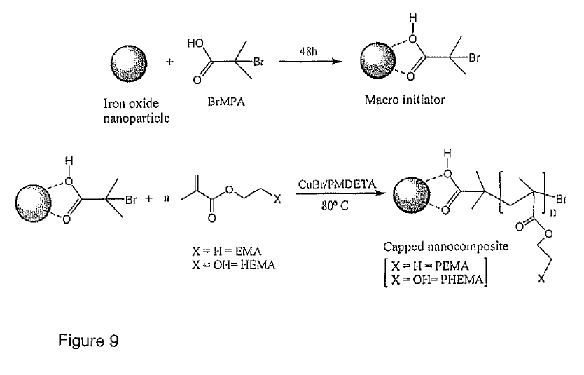

FIG. 10: MRI images which show the convection (drag) of the protein-decorated polyethyleneglycol capped magnetic nanoparticles in the gradient field of the MRI.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

An important step towards successful drug targeting with nanoparticles is developing a method to coat the nanoparticles with a useful drug. BSA was used by us to mimic the actual drug for its cost effectiveness during initial trials. As will be appreciated by one of skill in the art and as discussed herein, these results indicate that a drug of interest, that is, a pharmaceutical composition or compound or a bioactive protein, bioactive drug or bioactive drug protein can be loaded or coated onto the iron oxide nanocomposites. We have successfully immobilized BSA onto three (PEG, PEMA and glutamic acid) out of four different surfactant capped nanocomposites. These polymers are selected for their biocompatibility. Poly(ethylene glycol) PEG is a well established biocompatible polymer. It is known to be a non-toxic, non-immunogenic, non-antigenic and protein-resistant polymer. It can therefore increase the blood half-life of the nanoparticles. Studies carried out using PEMA has shown it to be biocompatible and having the potential to act as a drug carrier. PHEMA based polymers are widely used hydrogels in pharmaceutical applications. While not wishing to be bound to a specific theory, it is believed that PHEMA forms a hydrogel and has ‘groves’ which are nanosized. PHEMA can soak up water and swell. In the process, there is a possibility of pulling in and immobilizing the protein. A possible explanation is that PHEMA coating after absorbing water forms extensive hydrogen bond network and becomes insoluble in water, which prevents the protein from attaching to the polymer.

We found that the BSA immobilized particles showed excellent colloidal stability in water and stayed well suspended without any sign of agglomeration or settling. The suspended particles were easily accumulated using a magnet and could be re-dispersed readily. These properties indicate that the BSA immobilized iron oxide nanocomposites are excellent candidates for directed drug convection (DDC).

As discussed herein, in one aspect of the invention, there is provided a method of preparing surface modified ion oxide nanocomposites comprising:

dissolving FeCl₃.6H₂O and FeCl₂.4H₂O in heated water comprising a suitable biocompatible polymer;

adding ammonium hydroxide and mixing, thereby producing a mixture;

cooling the mixture to about room temperature; and

recovering surface modified iron oxide nanocomposites from the mixture.

The suitable biocompatible polymer may be selected from the group consisting of polyethylene glycol (PEG), poly(ethyl methacrylate) (PEMA) and glutamic acid.

The surface modified iron oxide nanocomposites may have a diameter of approximately 7-20 nanometers.

The FeCl₃.6H₂O and the FeCl₂.4H₂O may be mixed at approximately a 2:1 ratio.

The water may be heated to about 80° C.

In another aspect of the invention, there are provided recovered or purified surface modified iron oxide nanocomposited prepared as described herein.

As will be appreciated by one of skill in the art, for medical applications it is essential to synthesize smaller nanoparticles (7-20 nm), and it is essential for the particles to be stable under physiological conditions (pH about 7). Also, the particles should be undetectable by the immune system.

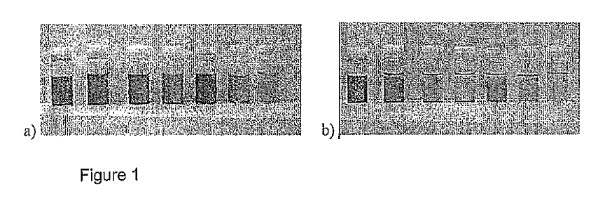

Physical properties such as appearance and colloidal stability of the particles in different solvents changes considerably with surface modification (FIG. 1). The bare iron oxide nanoparticles could be dispersed with sonication and complete settlement does not take place in a week. The PEG modified magnetite nanoparticles were easily dispersed in water and near complete settlement of particles took place within one week. The glutamic acid coated nanocomposites dispersed in water completely settled within a time span of one week. The PEMA capped particles remained well dispersed in the water and complete settlement did not take place even after a week. However, the PHEMA capped particles showed very poor dispersability in water and settled down very quickly within a matter of a few hours. Of all the particles, the BSA immobilized particles showed the highest solubility in water and very little settling took place over one week (FIG. 1).

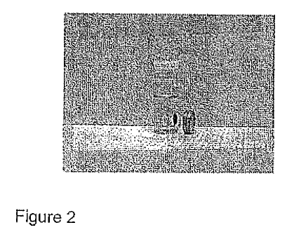

As shown in FIG. 2, the water dispersed PEMA capped iron oxide nanocomposites were readily attracted to the wall of the sample vial using a rare earth magnet. This shows that these nanocomposites dispersed in water are still magnetic and can be controlled by a magnetic field.

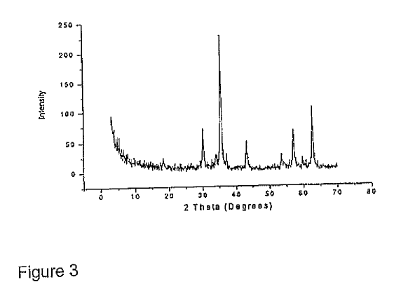

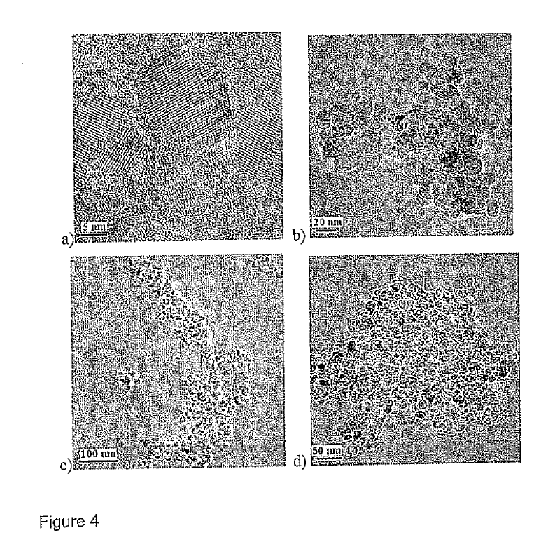

HR-TEM, XRD and SEM were carried out to characterize the microstructure of the as-synthesized nanoparticles. The XRD pattern obtained for the particles suggested the presence of iron oxide with a pattern characteristically related to magnetite (Fe₃O₄) and maghemite (ã-Fe₂O₃). FIG. 3 shows the XRD diffraction pattern of the bare iron oxide nanoparticles and the average particle diameter obtained using the Scherrer formula was 11 nm. For HR-TEM images, the particles were dispersed in a suitable quick drying solvent like acetone or Dichloromethane. The dispersed solution was dropped onto a carbon coated copper grid (400 mesh) and allowed to dry quickly. HR-TEM images (FIG. 4) clearly show that the nanoparticies are crystalline in nature (see atomic lattice fringes in FIG. 4a). Most of the individual coated nanocomposite can be easily distinguished from surrounding particles in these images. The particle diameters calculated from HR-TEM images are in the range of 11-13 nm and are in good agreement with XRD results.

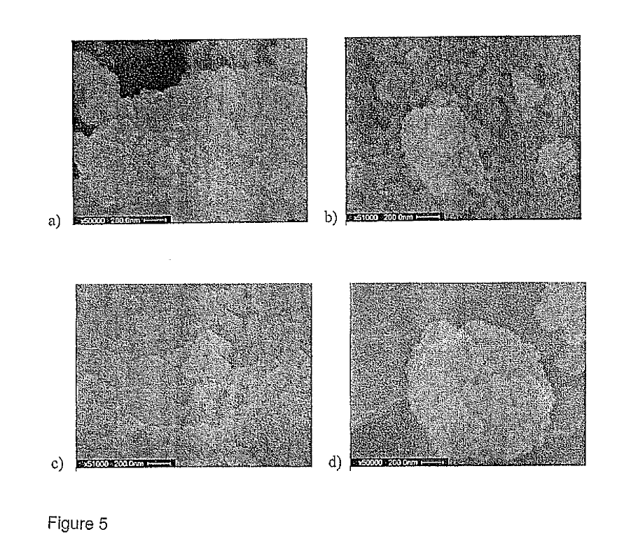

FIG. 5 shows the SEM images of surface modified iron oxide nanocomposites. The amorphous nature of the polymer-capped nanoparticles is clearly visible. All the particles were attached to a carbon tape before being observed using the SEM.

The colloidal stability of all the particles was studied using dynamic light scattering experiments (DLS). The hydrodynamic radius for the BSA immobilized particles were the lowest at 80 nm as compared to 150 nm to 2 micron for the aggregated, polymer coated nanocomposites without immobilized protein. From this study it is clear that the BSA immobilized iron oxide nanocomposites are significantly dispersible in water. This property is a must if these particles are to be administered as a drug carrier intravenously.

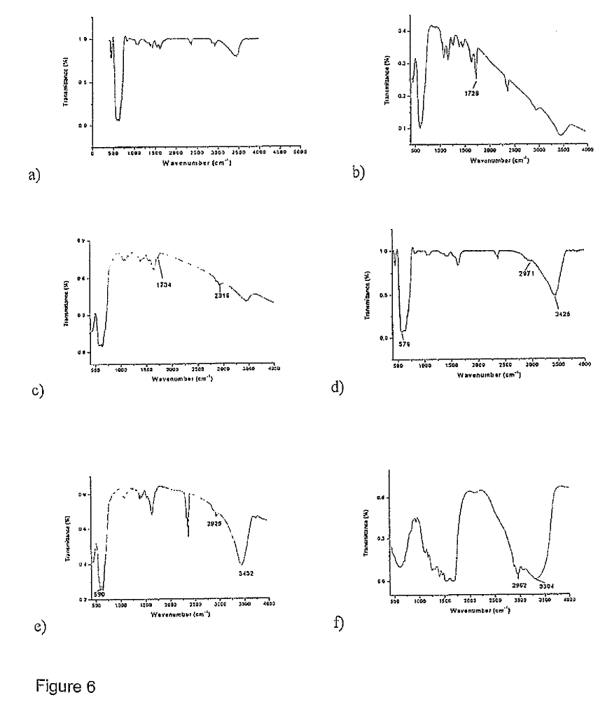

FT-IR spectra obtained from the as-synthesized nanoparticles clearly show that the iron oxide nanoparticles are surface modified (FIG. 6). A prominent peak at around 580 cm⁻¹ corresponds to Fe—O stretching in iron oxide. The peaks at 1726 cm⁻¹ and 1734 cm⁻¹ in FIG. 6b and 6c can be assigned to carbonyl (C═O) stretching in PEMA and PHEMA. The peak around 2900 cm⁻¹ can be assigned to aliphatic C-H stretching. The characteristic peaks corresponding to organic surfactant molecules along with a peak at around 580 cm⁻¹ in the IR spectra show clearly that these molecules are attached to iron oxide nanoparticle.

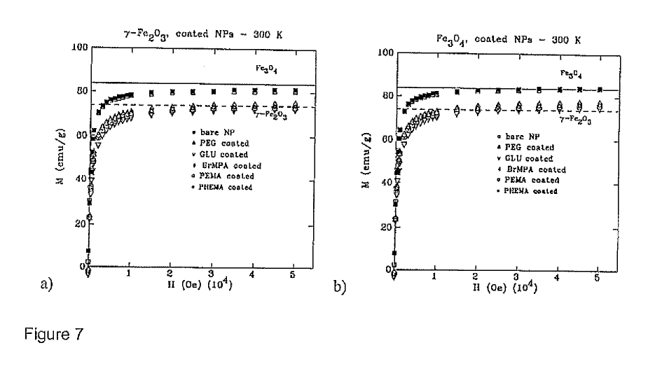

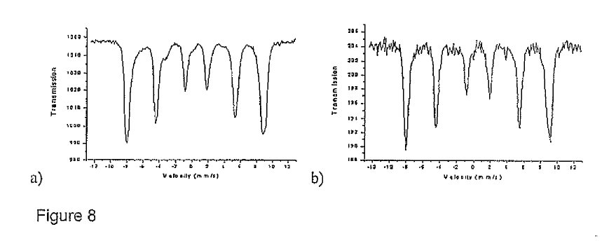

The room temperature (300 K) magnetic studies of the particles were carried out using a magnetometer. The magnetization of the particles was obtained with increasing magnetic field (H). FIGS. 7a and 7b show the field-dependent magnetization curve of different particles weight normalized with respect to ã-Fe₂O₃ and Fe₃O₄ respectively. From the two plots it can be observed that the bare iron oxide and the PHEMA capped nanocomposite consist of the iron oxide magnetite Fe₃O₄ (shown as a solid line at 82 emu/g). The PEG, glutamic acid, BrMPA and PEMA capped iron oxide nanocomposites consist of the iron oxide maghemite ã-Fe₂O₃ (shown as dotted line at 76 emu/g). The Mössbauer spectra (FIG. 8) that measure the atomic magnetism of the Fe₃O₄ and ã-Fe₂O₃ core nanocomposites confirm the magnetometry results.

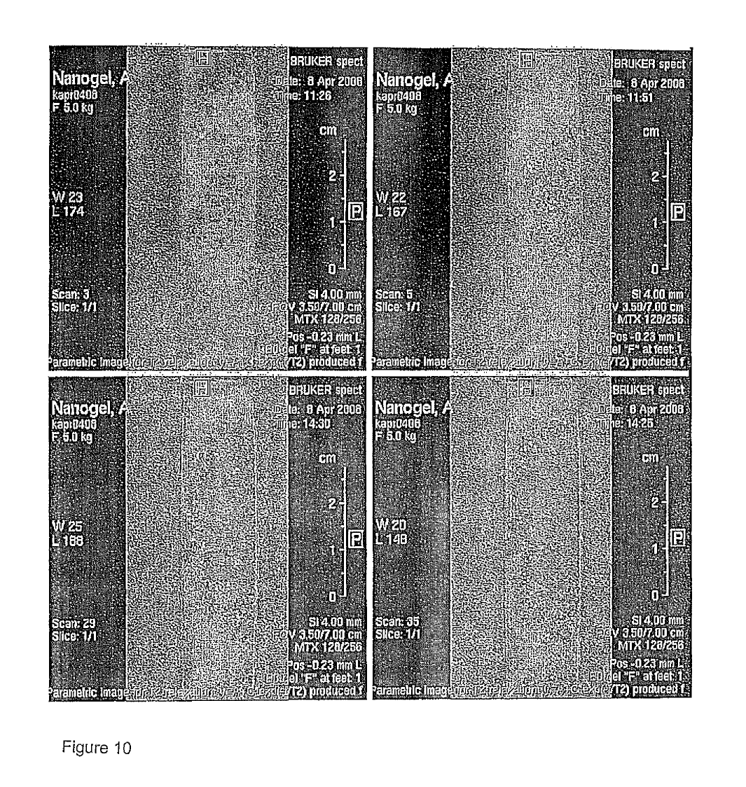

FIG. 10 shows a number of MRI images showing the convection (drag) of the protein-decorated polyethyleneglycol capped magnetic nanoparticles in the gradient field of the MRI. The first two images are about 30 minutes apart. The third image was acquired 1 hour later and the final image was acquired 2 hours later. As will be appreciated by one of skill in the art, this experiment proves how the protein loaded magnetic carriers can be used for directed drug delivery and simultaneous MRI imaging as well as for ‘clot-busting’ with simultaneous MRI imaging if desired.

We have gained significant experience in synthesizing iron oxide nanoparticles with a consistent and narrow size distribution. We have synthesized iron oxide nanocomposites of around 11-13 nm in diameter coated with different biocompatible surfactant molecules. We have further immobilized BSA onto these surface modified iron oxide nanocomposites and learned that the nature of the surfactant molecules is crucial for immobilizing a particular protein. PEG, glutamic acid and PEMA were identified as suitable surfactant molecules for immobilizing BSA. PHEMA was not suitable for immobilizing BSA.

These results are extendable to other bioactive drug proteins. The next step will be to attach the tissue plasminogen activator (t-PA) to the PEG, glutamic acid and PEMA capped iron oxide core nanocomposites. We will quantify the amount of protein immobilized onto the core/shell magnetic nanocomposites and evaluate effective delivery of the drug and its mobility ex-vivo and in-vivo. We are also in the process of testing other biocompatible polymers as surfactants, and modifying the iron oxide core size below 10 nm to study the dependence of the nanoparticle size on the magnetic properties of the final, drug-immobilized magnetic nanocomposite.
Decorating with active drug (r-tPA)
Establishing simultaneous mass transport and visualization using 7T MRI in agarose and or alginate gels
Manipulating outer shell to adjust zeta-potential (overall surface charge of the particle)
Ex-vivo mass transport in simple and complex (blood-like) fluids
Different medically active proteins and drugs (crossing blood-brain barrier, etc.)

### Directed Drug Delivery: Ex-Vivo Mass Transport

To guide the drug coated magnetic nanoparticles through complex media such as tissue requires static and oscillating magnetic fields. With static magnetic field gradients, magnetic nanoparticles can be attracted to a location in simple fluids. However, in complex media, the particles get stuck on their way. An oscillatory magnetic field can overcome this mechanical arrest, like a gopher burrowing through sand, enabling further diffusion of the nanoparticle through the media. A drug delivery targeting system will involve a static field provided by Helmholtz coils that have a constant electric current running through their coil windings, and a small oscillating magnetic field that is superposed on the static field by way of a separate coil that has a field provided by an alternating current.

### Synthesis of Iron Oxide Nanoparticles

The iron oxide nanoparticles were prepared following a co-precipitation method. 8 mmol of FeCl₃6H₂O and 4 mmol of FeCl₂4H₂O were dissolved in 200 ml of deionised water (DI) (R=18 MU) under a nitrogen atmosphere. The solution was stirred for 15 minutes using an overhead stirrer. The temperature of the reaction mixture was maintained at about 80° C. 24 ml of aqueous NH₄OH (14-15%) were added drop-wise to the reaction vessel accompanied by vigorous stirring (600 rpm). During the process, the colour of the reaction mixture turned from orange to black. The formation of magnetite nanoparticles takes place in the pH range of about 7.5-14. The black magnetite powder obtained was washed three times with DI water. Thereafter, the water was removed under reduced pressure, and the nanoparticles were dried under vacuum.

### Synthesis of Peg and Glutamic Acid Surface Modified Magnetite Nanoparticles:

These surface modified magnetite nanoparticles were prepared following a co-precipitation method in the presence of the surfactant molecules. An approximately 2:1 mmol ratio of FeCl₃6H₂O and FeCl₂4H₂O was added to deionized and deoxygenated water at about 80° C. containing 2.0 g of PEG for the PEG modified and 1.77 g of glutamic acid for the amino acid coated iron oxide nanoparticles. To the above mixtures, a suitable amount, for example, about 12 ml of aqueous ammonium hydroxide (about 14-15%) were added drop-wise with vigorous stirring (600 rpm). After a while, the entire solution turned black. This mixture was stirred at about 80° C. for approximately one hour, followed by approximately three hours of additional stirring at room temperature. As will be appreciated by one of skill in the art, other suitable mixing times may be used and can be readily determined through routine experimentation. The reaction mixture was then washed three times with DI water using sonication, followed by centrifugation at 4000 rpm. The resulting surface modified iron oxide nanocomposites were dried under vacuum overnight.

### Synthesis of BrMPA Functionalized Iron Oxide Nanocomposites:

2-Bromo-2-methyl propionic acid (BrMPA) attached to iron oxide nanoparticles acts as a macro-initiator (Scheme 1) for atom transfer radical polymerization (ATRP) to graft poly(ethyl methacrylate) PEMA and poly(2-hydroxyethyl methacrylate) PHEMA on to the nanoparticles. 1 mmol of nanoparticles was added to 10 ml of hexane and sonicated for 15 minutes. To that, 0.72 mmol of BrMPA was added, and the solution was sonicated for an additional 5 minutes. The mixture was then stirred for 48 hours at room temperature. After completion of the reaction, 20 ml of ethanol was added. A brown precipitate was obtained which was then collected by centrifugation at 4000 RPM for 15 minutes. The residue obtained was washed three times using hexane under sonication to remove any unattached initiator molecules.

### Synthesis of Poly(Ethyl Methacrylate) PEMA and Poly(2-Hydroxyethyl Methacrylate) PHEMA Functionalized Iron Oxide Nanocomposites (Scheme 1):

BrMPA functionalized iron oxide nanocomposites (0.996 g), copper(I)bromide (CuBr, 90.0 mg, 0.63 mmol), N,N,N˜,N˜,N˜-pentamethyl diethylenetriamine (PMDETA, 150 mg, 0.87 mmol), freshly distilled ethyl methacrylate (EMA, 1.5 g, 13.2 mmol) and anisole (4.5 g) were suspended in a 100 ml thick walled round bottom flask. For PHEMA capped nanocomposites, 2-hydroxyethyl methacrylate (HEMA) was used in the place of EMA. All the contents were subjected to three freeze-pump-thaw cycles to degas the mixture. Once the contents attained room temperature, they were heated to 85° C. and stirred at this temperature for three hours. The reaction mixture was diluted with tetrahydrofuran THF (10 times the volume of the reaction mixture). Thereafter, the mixture was precipitated with methanol. The brown precipitate was centrifuged at 4000 rpm, and the residue was washed three times with dichloromethane and ethanol. Finally, the product was dried at room temperature under vacuum.

Immobilization of Bovine Serum Albumin (BSA) onto the Iron Oxide Nanocomposites

The surface modified iron oxide nanocomposites were sonicated in DI water until the particles were homogeneously dispersed. Sonication was continued for an additional 10 minutes. To this solution, BSA (in this example, about 10 times the weight of nanocomposite although other suitable ratios may be used) was added and sonicated for 15 minutes. After sonication, the mixture was subjected to vigorous stirring (1200 rpm) for three hours. The product was then centrifuged for 10 minutes at 4000 rpm. The final BSA-immobilized particles would not settle down in solution. The solution was collected and the particles were subjected to magnetic separation. The isolated particles were then washed twice with water, and dried under vacuum. However, PHEMA coated magnetite nanoparticles could not be used to immobilize BSA using the above mentioned procedure.

As will be appreciated by one of skill in the art, many of the incubation times and ratios listed above represent ‘minimums’ and longer periods of time and/or greater amounts may be used where appropriate.

While the preferred embodiments of the invention have been described above, it will be recognized and understood that various modifications may be made therein, and the appended claims are intended to cover all such modifications which may fall within the spirit and scope of the invention.

### REFERENCES

(1) (a) Braehler, M.; Georgieva, R.; Buske, N.; Mueller, A.; Mueller, S.; Pinkernelle, J.; Teichgraeber, U.; Voigt, A.; Baeumler, H., Magnetite-Loaded Carrier Erythrocytes as Contrast Agents for Magnetic Resonance Imaging. Nano Lett. 2006, 6(11), 2505-2509; (b) Ai, H.; Flask, C.; Weinberg, B.; Shuai, X.; Pagel, M. D.; Farrell, D.; Duerk, J.; Gao, J., Magnetite-loaded polymeric micelles as ultrasensitive magnetic-resonance probes. Adv. Mater. 2005, 17(16), 1949-1952.
2 (a) Gordon, R. T.; Gordon, D., Resonance induced alterations of intracellular biophysical properties. Med. Hypotheses 1979, 5(9), 1025-35; (b) Chan, D. C. F.; Kirpotin, D. B.; Bunn, P. A. Jr., Synthesis and evaluation of colloidal magnetic iron oxides for the site-specific radiofrequency-induced hyperthermia of cancer. J. Magn. Magn. Mater. 1993, 122(1-3), 374-378; (c) Kawashita, M.; Tanaka, M.; Kokubo, T.; Inoue, Y.; Yao, T.; Hamada, S.; Shinjo, T., Preparation of ferromagnetic magnetite microspheres for in situ hyperthermic treatment of cancer. Biomaterials. 2005, 26(15), 2231-2238.
3 Bai, Y.; Teng, B.; Chen, S.; Chang, Y.; Li, Z., Preparation of magnetite nanoparticles coated with an amphiphilic block copolymer: a potential drug carrier with a core-shell-corona structure for hydrophobic drug delivery. Macromol. Rapid Commun. 2006, 27(24), 2107-2112.
4 Mornet, S.; Vasseur, S.; Grasset, F.; Duguet, E., Magnetic nanoparticle design for medical diagnosis and therapy. J. Mater. Chem. 2004, 14(14), 2161-2175.
5 (a) Flesch, C.; Unterfinger, Y.; Bourgeat-Lami, E.; Duguet, E.; Delaite, C.; Dumas, P., Poly(ethylene glycol) surface coated magnetic particles. Macromol. Rapid Commun. 2005, 26(18), 1494-1498; (b) Wan, S.; Zheng, Y.; Liu, Y.; Yan, H.; Liu, K., Fe3O4 nanoparticles coated with homopolymers of glycerol mono(meth)acrylate and their block copolymers. J. Mater. Chem. 2005, 15(33), 3424-3430; (c) Yuan, J.-J.; Armes, S. P.; Takabayashi, Y.; Prassides, K.; Leite, C. A. P.; Galembeck, F.; Lewis, A. L. Synthesis of Biocompatible Poly[2-(methacryloyloxy)ethyl phosphorylcholine]-Coated Magnetite Nanoparticles. Langmuir 2006, 22(26), 10989-10993.
6 (a) Okubo, M.; Azume, I.; Yamamoto, Y. Preferential adsorption of bovine serum albumin dimer onto polymer microspheres having a heterogeneous surface consisting of hydrophobic and hydrophilic parts. Colloid. Polym. Sci. 1990, 268(7), 598-603; (b) Zhang, C.; Cao, J.; Yin, D.; Wang, Y.; Feng, Y.; Tan, J., Preparation and radiolabeling of human serum albumin (HSA)-coated magnetite nanoparticles for magnetically targeted therapy. Appl. Radiat. Isot. 2004, 61(6), 1255-1259; (c) Macaroff, P. P.; Simioni, A. R.; Lacava, Z. G. M.; Lima, E. C. D.; Morais, P. C.; Tedesco, A. C., Studies of cell toxicity and binding of magnetic nanoparticles with blood stream macromolecules. J. Appl. Phys. 2006, 99(8, Pt. 3); (d) Pan, B.-F.; Gao, F.; Gu, H.-C., Dendrimer modified magnetite nanoparticles for protein immobilization. J. Colloid Interface Sci. 2005, 284(1), 1-6. (e) Pan, B.-F.; Gao, F.; Ao, L.-M., Investigation of interactions between dendrimer-coated magnetite nanoparticles and bovine serum albumin. J. Magn. Magn. Mater. 2005, 293(1), 252-258; (f) Ma, Z.; Guan, Y.; Liu, X.; Liu, H., Covalent immobilization of albumin on micron-sized magnetic poly(methyl methacrylatedivinylbenzene-glycidyl methacrylate) microspheres prepared by modified suspension polymerization. Polym. Adv. Technol. 2005, 16(7), 554-558.
7 (a) Xu, X. Q.; Shen, H.; Xu, J. R.; Xie, M. Q.; Li, X. J. The colloidal stability and core-shell structure of magnetite nanoparticles coated with alginate. Appl. Surf. Sci. 2006, 253(4), 2158-2164; (b) Zhang, L.; He, R.; Gu, H.-C., Oleic acid coating on the monodisperse magnetite nanoparticles. Appl. Surf. Sci. 2006, 253(5), 2611-2617; (c) Kim, D. K.; Mikhaylova, M.; Zhang, Y.; Muhammed, M., Protective Coating of Superparamagnetic Iron Oxide Nanoparticles. Chem. Mater. 2003, 15(8), 1617-1627.

1. A method of preparing surface modified ion oxide nanocomposites comprising:
dissolving FeCl₃.6H₂O and FeCl₂.4H₂O in heated water comprising a suitable biocompatible polymer;
adding ammonium hydroxide and mixing, thereby producing a mixture;
cooling the mixture to about room temperature; and
recovering surface modified iron oxide nanocomposites from the mixture.

2. The method according to claim 1 wherein the suitable biocompatible polymer is selected from the group consisting of polyethylene glycol (PEG), poly(ethyl methacrylate) (PEMA) and glutamic acid.

3. The method according to claim 1 wherein the surface modified iron oxide nanocomposites have a diameter of approximately 7-20 nanometers.

4. The method according to claim 1 wherein the FeCl₃.6H₂O and the FeCl₂.4H₂O are mixed at approximately a 2:1 ratio.

5. The method according to claim 1 wherein the water is heated to about 80° C.

6. The method according to claim 1 including adding a drug to the recovered surface modified iron oxide nanocomposites, stirring and recovering drug coated surface modified iron oxide nanocomposites.

7. Surface modified iron oxide nanocomposited prepared according to the method of claim 1.
